(19) United States
(12) Patent Application Publication
(10) Pub. No.: US 2010/0292482 A1
(43) Pub. Date: Nov. 18, 2010

(54) NOVEL PRECURSORS

(75) Inventors: David Alexander Learmonth, Valongo (PT); Laszlo Erno Kiss, Lavra (PT)

Correspondence Address: FOLEY & LARDNER LLP, 111 HUNTINGTON AVENUE, 26TH FLOOR, BOSTON, MA, 02199-7610, US

(73) Assignee: BIAL -Portela & CA.., S.A.

(21) Appl. No.: 12/739,669

(22) PCT Filed: Oct. 24, 2008
(86) PCT No.: PCT/PT08/00042

§ 371 Date: Aug. 6, 2010

(30) Foreign Application Priority Data
Oct. 24, 2007 (GB) 0720866.3
Feb. 26, 2008 (EP) 08003466.3

(51) Int. Cl. C07D 213/12; C07C 229/30; C07C 255/17; C07D 213/80; C07D 213/84; C07D 213/78

(52) U.S. Cl. 546/250; 562/567; 558/440; 546/318; 546/286; 546/332

(57) ABSTRACT

The present invention relates to a Compound of the formula (I), wherein X represents CN, COOR, wherein R represents hydrogen or a carboxyl protecting group, CONR′2, wherein R′ represents hydrogen or a carboxyl protecting group, or nitro; R1, R2, R3, R4, R5 independently of each other represent hydrogen, C1-C6 alkyl, halogen, trifluoromethyl, cyano, nitro, substituted aryl or substituted heteroaryl group; Y represents hydrogen, C1-C6 alkyl, C3-C7 cycloalkyl, C7-C13 alkaryl, trifluoromethyl, cyano, nitro, substituted aryl or substituted heteroaryl group; and wherein the stereochemically unspecified double bonds in the above formula (I) represent either the E,E; E,Z; Z,E or Z,Z configuration.

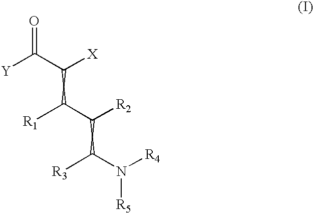

This invention relates to novel precursors of an amidoxime building block for the synthesis of highly potent, long-acting peripheral inhibitors of catechol-O-methyl-transferase (COMT) and to processes for the preparation of said precursors.

COMT inhibitors are used as adjuncts to L-DOPA/peripheral amino acid decarboxylase (AADC) inhibitor therapy in patients afflicted by Parkinson's disease. Use of COMT inhibitors is based on their ability to reduce metabolic O-methylation of L-DOPA to 3-O-methyl-L-DOPA (3-OMD). Thus, L-DOPA is protected from metabolic breakdown, its mean plasma concentration is raised and its bioavailability is increased. Well-known COMT inhibitors such as Tolcapone, and Entacapone show disadvantages such as liver damage, short in-vivo half-lives or undesired central nervous system side-effects.

Potent novel peripheral COMT inhibitors based on nitrocatechol derivatives have been described in the international patent application WO 2007/013830 which is entirely incorporated herein by reference. As described in the same application, the non-catecholic substituents of said derivatives obtained by employing a trifluoromethylated amidoxime building block determine the lack of toxic effects of the compounds. An example of such a COMT inhibitor based on a nitrocatechol derivative is shown below.

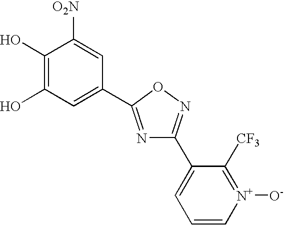

In view of the above, it is the object of the present invention to provide novel processes and corresponding precursors that enable a more efficient synthesis of COMT inhibitors based on nitrocatechol derivatives.

This object has been achieved by the finding of surprisingly efficient and versatile pathways and novel precursors to trifluoromethylated amidoxime building blocks.

In one aspect, the present invention relates to a synthetic pathway utilizing novel precursors of the following general formula (I):

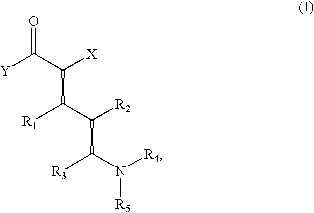

wherein X represents CN, COOR, wherein R represents hydrogen or a carboxyl protecting group, CONR′₂, wherein R′ represents hydrogen or C₁-C₆ alkyl, or nitro; R₁, R₂, R₃, R₄, R₅ independently of each other represent hydrogen, C₁-C₆ alkyl, halogen, trifluoromethyl, cyano, nitro, substituted aryl or substituted heteroaryl group; Y represents hydrogen, C₁-C₆ alkyl, C₃-C₇ cycloalkyl, C₇-C₁₃ alkaryl, trifluoromethyl, cyano, nitro, substituted aryl or substituted heteroaryl group; and wherein the stereochemically unspecified double bonds in the above formula (I) represent either the E,E; E,Z; Z,E or Z,Z configuration.

In a preferred embodiment of the present invention, in the above formula (I), Y represents trifluoromethyl and X represents CN or COOR, wherein R represents hydrogen, C₁-C₆ alkyl, C₁-C₆ haloalkyl, C₆-C₁₂ aryl, C₇-C₁₃ aralkyl or C₇-C₁₃ alkaryl; and R₁ to R₃ represent hydrogen and R₄ and R₅ represent methyl groups.

In a more preferred embodiment of the present invention, in the above formula (I), Y represents trifluoromethyl and X represents CN or COOR, wherein R represents methyl, trifluoromethyl, diphenylmethyl, ethyl, trichloroethyl, propyl, iso-propyl, butyl, tert-butyl, phenyl, or benzyl; and R₁ to R₃ represent hydrogen and R₄ and R₅ represent methyl groups.

In the most preferred embodiment of the present invention, in the above formula (I), Y represents trifluoromethyl and X represents CN or COOR, wherein R represents ethyl; and R₁ to R₃ represent hydrogen and R₄ and R₅ represent methyl groups.

The present invention also relates to two processes for the preparation of compounds of the general formula (I).

The first process for the preparation of compound of the general formula (I) comprises a reaction between a compound of formula (II),

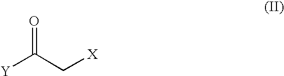

wherein X and Y are as defined in formula (I), and a compound of formula (III),

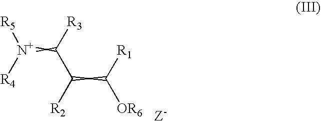

wherein R₁, R₂, R₃, R₄ and R₅ are defined as in formula (I), R₆ is C₁-C₆ alkyl; and Z⁻ represents a suitable counter ion, such as Cl⁻ or POCl₂⁻; and wherein the stereochemically unspecified double bonds in the above formula (III) represent the E,E; E,Z; Z,E or Z,Z configuration.

Preferably in the above formula (II), Y represents trifluoromethyl and X represents CN or COOR, wherein R represents hydrogen, C₁-C₆ alkyl, C₁-C₆ haloalkyl, C₃₋₇ cycloalkyl, C₆-C₁₂ aryl, C₇-C₁₃ aralkyl or C₇-C₁₃ alkaryl.

More preferably in the above formula (II), Y represents trifluoromethyl and X represents CN or COOR, wherein R represents methyl, trifluoromethyl, diphenylmethyl, ethyl, trichloroethyl, propyl, iso-propyl, butyl, tert-butyl, phenyl, or benzyl.

Most preferably in the above formula (II), Y represents trifluoromethyl and X represents CN or COOR, wherein R represents ethyl.

Preferably in the above formula (III), R₁ to R₃ are hydrogen atoms, R₄ and R₅ are methyl groups and R₆ is a n-butyl group.

Alternatively, the first process of the invention utilizes a compound of formula (III), wherein R₁ to R₃ are hydrogen atoms, R₄ and R₅ are methyl groups and R₆ is a butyl group, and a compound of formula (II), where X is CN or COOR, where R is methyl, trifluoromethyl, diphenylmethyl, ethyl, trichloroethyl, propyl, iso-propyl, butyl, tert-butyl, phenyl or benzyl; and where Y is trifluoromethyl.

An alternative more preferred embodiment of the first process of the invention utilizes a compound of formula (III), wherein R₁ to R₃ are hydrogen atoms, R₄ and R₅ are methyl groups and R₆ is a butyl group, and a compound of formula (II), where X is CN or COOR, where R is ethyl and Y is trifluoromethyl.

The reaction between compounds of formula (II) and (III) occurs preferably in the presence of a base. A suitable base according to the present invention is a non-nucleophilic base such as trimethylamine, triethylamine, diisopropylethylamine, pyridine, lutidine, N,N-dimethyl piperidine, N-methyl-pyrrolidine, 1,5-diazabicyclo[4,3,0]non-5-ene, 1,8-diazabicyclo[5,4,0]undec-7-ene or sodium hydride is used.

Preferably the reaction occurs in the presence of an organic solvent, for example DCM.

The immonium ion of formula (III) may be prepared in a Vilsmeier-Haack recation by reacting N,N-dimethylformamide with an activating agent such as oxalyl chloride thionyl chloride or phosphorous oxychloride in an organic solvent followed by addition of an optionally substituted 1-vinyloxyalkane, for example n-butyl-vinylether at a temperature of between 0 and 10° C., preferably at 5° C. Suitable counterions Z⁻ are known in the art and include Cl⁻ or POCl₂⁻. The compound of formula (II) is preferably added to the solution at a temperature between −20° C. and 20° C., preferably −5° C. and 3° C., to form N-4-carboxy-6,6,6-trifluoro-5-hydroxyhexa-2,4-dienylidene)-N-methylmethanaminium chloride derivative. The compound of formula (I) was then created by quenching with an aqueous solution of a mineral acid such as HCl, H₂SO₄, or H₃PO₄. In a preferred embodiment the acid is HCl. Preferably the quenching step was performed at a temperature of from 0° C. to 20° C., more preferably from 5° C. to 10° C., most preferably at 8° C.

The organic solvent employed in this reaction should preferably be inert to the Vilsmeier-reagent. Suitable solvents may be selected from dichloromethane, 1,2-dichloroethane, 1,1,2,2-tetrachloroethane or chlorobenzenes or mixtures thereof.

In the above process, the stereochemistry of the educts and intermediates is of less importance since the stereochemistry is equilibrated in, the course of the reaction. Preferably, the stereochemistry of the obtained compound of formula (I) is such that the substituents R₁ and X as well as R₂ and R₃ are each in trans-positions. However, the compounds having the cis-positions as well as mixtures (i.e. mixtures of E,E-, E,Z-, Z,E- and Z,Z-isomers) are also acceptable.

The second process for the preparation of compound (I) comprises a reaction of a compound of the above formula (II), wherein X represents CN: COOR, wherein R represents hydrogen or a carboxyl protecting group: CONR′2, wherein R′ represents hydrogen or C₁-C₆ alkyl, C₃₋₇ cycloalkyl or C₇₋₁₃ alkaryl: or nitro; and Y represents hydrogen, C₁-C₆ alkyl, C₁-C₆ haloalkyl e.g. trifluoromethyl, C₃-C₇ cycloalkyl, C₇-C₁₃ alkaryl, cyano, nitro, substituted aryl or substituted heteroaryl group, with a compound of formula (IV),

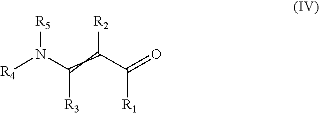

wherein R₁ represents hydrogen, R₂, R₃, R₄, R₅ independently of each other represent hydrogen, C₁-C₆ alkyl, halogen, trifluoromethyl, cyano, nitro, substituted aryl or substituted heteroaryl group.

Preferably in the second process of the present invention, in the formula (II), Y represents trifluoromethyl and X represents CN or COOR, wherein R represents hydrogen, C₁-C₆ alkyl, C₁-C₆ haloalkyl, C₆-C₁₂ aryl, C₇-C₁₃ aralkyl or C₇-C₁₃ alkaryl.

More preferably in the second process of the present invention, in the above formula (II), Y represents trifluoromethyl and X represents CN or COOR, wherein R represents methyl, trifluoromethyl, diphenylmethyl, ethyl, trichloroethyl, propyl, iso-propyl, butyl, tert-butyl, phenyl, or benzyl.

Most preferably in the second process of the present invention, in the above formula (II), Y represents trifluoromethyl and X represents CN or COOR, wherein R represents ethyl.

Preferably in the present invention, in the above formula (IV), R₁ to R₃ are hydrogen atoms, and R₄ and R₅ are methyl groups.

In an alternative preferred embodiment the second process for the preparation of compounds of the general formula (I) utilizes a compound of formula (IV), wherein R₁ to R₃ are hydrogen atoms, and R₄ and R₅ are methyl groups, and a compound of formula (II), wherein X is CN or COOR, wherein R is methyl, trifluoromethyl, diphenylmethyl, ethyl, trichloroethyl, propyl, iso-propyl, butyl, tert-butyl, phenyl or benzyl; and where Y is trifluoromethyl.

In a more preferred alternative embodiment, the second process for the preparation of compounds of the general formula (I) utilizes a compound of formula (IV), wherein R₁ to R₃ are hydrogen atoms, and R₄ and R₅ are methyl groups and a compound of formula (II), wherein X is CN or COOR, wherein R is ethyl and Y is trifluoromethyl.

The reaction between compounds of formula (II) and (IV) is preferably carried out in the presence of an activating agent. Exemplary activating agents include acetic anhydride, trifluoroacetic anhydride, methylsulfonic anhydride, phenylsulfonic anhydride, succinic anhydride, phthalic anhydride or an acid chloride. In a preferred embodiment of the invention, the activating agent is acetic anhydride. In addition to the activating agent, a non-nucleophilic base such as trimethylamine, triethylamine, diisopropylethylamine, pyridine, lutidine, N,N-dimethyl piperidine, N-methyl-pyrrolidine, 1,5-diazabicyclo[4.3.0]non-5-ene, 1,8-diazabicyclo[5.4.0]undec-7-ene or sodium hydride can be used in the preparation of compounds of formula (I). Preferably the reaction is carried out in the absence of a base. The reaction is preferably carried out at a temperature between 0° C. and 100° C., preferably at ambient temperature.

The reaction between compounds of formula (II) and (IV) may result in the in situ formation of the corresponding intermediate enolacetate.

The present invention further relates to a process for the preparation of compounds of formula (V),

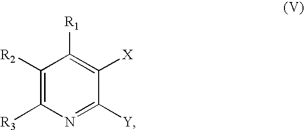

wherein X represents CN, COOR, wherein R represents hydrogen or a carboxyl protecting group such as C₁-C₆ alkyl, C₃₋₇ cycloalkyl, and C₇₋₁₃ alkaryl; CONR′2, wherein R′ represents hydrogen, C₁-C₆ alkyl, C₃₋₇ cycloalkyl or C₇₋₁₃ alkaryl; or nitro; R₁, R₂, and R₃ independently of each other represent hydrogen, C₁-C₆ alkyl, halogen, trifluoromethyl, cyano, nitro, substituted aryl or substituted heteroaryl group; Y represents hydrogen, C₁-C₆ alkyl, halogen, C₃-C₇ cycloalkyl, C₇-C₁₃ alkaryl, trifluoromethyl, cyano, nitro, substituted aryl or substituted heteroaryl group; which comprises subjecting the compound of formula (I) as defined above to a cyclisation reaction.

A preferred embodiment of the cyclisation process of the invention uses a compound of formula (I) in which Y represents trifluoromethyl and X represents CN or COOR, wherein R represents hydrogen, C₁-C₆ alkyl, C₃₋₇ cycloalkyl, C₁-C₆ haloalkyl, C₆-C₁₂ aryl, C₇-C₁₃ aralkyl or C₇-C₁₃ alkaryl; and R₁ to R₃ represent hydrogen and R₄ and R₅ represent methyl groups.

A more preferred embodiment of this cyclisation process of the invention uses a compound of formula (I) in which Y represents trifluoromethyl and X represents CN or COOR, wherein R represents methyl, trifluoromethyl, diphenylmethyl, ethyl, trichloroethyl, propyl, iso-propyl, butyl, tert-butyl, phenyl, or benzyl; and R₁ to R₃ represent hydrogen and R₄ and R₅ represent methyl groups.

The most preferred embodiment of this cyclisation process of the invention uses a compound of formula (I) in which Y represents trifluoromethyl and X represents CN or COOR, wherein R represents ethyl; and R₁ to R₃ represent hydrogen and R₄ and R₅ represent methyl groups.

The cyclisation of a compound of formula (I) is preferably conducted in the presence of an ammonia source such as an ammonia solution in methanol, ethanol, isopropanol or butanol, ammonium acetate, ammonium sulfamate or ammonia which is directly introduced in gaseous form. The preparation of compound (V) is preferably carried out in a protic medium, preferably in an alcoholic medium, most preferably in methanol or water, and at temperatures between 60 and 70° C.

In an alternative embodiment, the reaction is carried out in the absence of water, e.g. by using dry solvents and gaseous ammonium.

The cyclisation of compounds of formula (I) yields pyridine derivatives which can be converted to compounds of formula (VI)

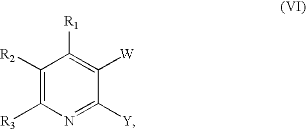

wherein R₁, R₂, R₃, and Y are as defined for formula (V) and W represents an amidoxime moiety (C(═N—OH)—NH₂). Such amidoxime-carrying compounds are important intermediates in the synthesis of oxadiazolyl-type COMT inhibitors. Therefore, the invention also makes available compounds of formula (VI) wherein W represents an amidoxime moiety via precursors in which W is CN, CONR′2, wherein R′ represents hydrogen or C₁-C₆ alkyl, or COOR, wherein R is as defined in formula (I).

Preferably, in formula (VI) Y is CF₃ and W is selected from CONH₂ and an amidoxime residue (C(═N—OH)—NH₂).

Oxadiazolyl-type COMT inhibitors are particularly effective if they comprise a pyridine-N-oxide structural element. Therefore, compounds of formula (VI) are also important precursors for compounds of formula (VII)

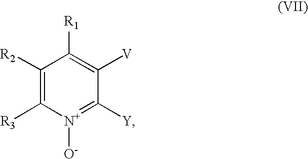

wherein R₁, R₂, R₃, and Y are as defined in formula (I), and V is CN, an amidoxime residue (C(═N—OH)—NH₂), nitro, CONR′2, wherein R′ represents hydrogen or C₁-C₆ alkyl, or COOR, wherein R is as defined as in formula (I).

Preferably, in formula (VII) Y represents CF₃ and V is selected from CN, CONH₂, an amidoxime residue (C(═N—OH)—NH₂) and COOR, wherein R is selected from hydrogen, methyl, ethyl, n-propyl, iso-propyl, n-butyl, tert-butyl, phenyl, benzyl, 4-nitro benzyl, 4-bromo benzyl, 4-methoxy benzyl, diphenylmethyl, and trichloroethyl.

The following examples illustrate the invention.

### EXAMPLE 1

### Preparation of Ethyl 5-(dimethylamino)-2-(2,2,2-trifluoroacetyl)-penta-2,4-dienoate

51.60 g (0.706 mol) of N,N-dimethylformamide was dissolved in 2 L of DCM in a 5 L three necked round bottom flask equipped with mechanical stirrer, thermometer, dropping funnel and N₂ inlet. The clear solution was cooled to 0° C. under N₂ atmosphere and oxalyl chloride (89.7 g, 61.7 ml 0.707 mol) was added dropwise over a period of to 30 min. During the addition a white precipitate was formed. The reaction mixture was allowed to warm up to room temperature and was stirred for 2 hours. It was cooled again to 0° C. and 1-(vinyloxy)butane (141.20 g, 182 ml) was added dropwise keeping the internal temperature below 5° C. The addition took 45 min. The cooling bath was then removed and the reaction was stirred for 2 hours under N₂. The pale yellow homogeneous solution was cooled again to −5° C., and ethyl trifluoro-acetoacetate (99.97 g, 0.543 mol) was added rapidly followed by the addition of triethylamine (157 g, 217 ml, 1.557 mol) keeping the temperature below 3° C. The cooling bath was removed and the red heterogeneous reaction mixture was stirred for 5 min. whereupon 750 ml of 1 N HCl was added to it below 8° C. The resultant two phase mixture was separated; the aqueous phase was extracted with 100 ml of DCM. The combined organic phases were washed twice with 300-300 ml of water and dried over MgSO₄. After filtration the solvent was removed by vacuum and evaporated again with 200 ml of toluene. The resulted red mobile oil was crystallized on standing overnight. The crude crystals were triturated with 100 ml of petroleum ether, filtered, washed with petroleum ether and dried in air.

Yield 80.60 g orange crystals (56%), mp:70° C.

### EXAMPLE 2

### Preparation of Ethyl 5-(dimethylamino)-2-(2,2,2-trifluoroacetyl)penta-2,4-dienoate

In a 20 ml pear flask was ethyl 4,4,4-trifluoro-3-oxobutanoate (3.00 g, 16.29 mmol), 3-(dimethylamino)acrylaldehyde (2.63 g, 26.6 mmol) was placed in acetic anhydride (5.8 ml) to give a brown solution. The reaction mixture was stirred for 10 min. TLC showed the reaction to be completed. The reaction mixture was dissolved in DCM. The organic phase was washed with 1 N HCl solution, water and then dried over MgSO₄. After filtration and evaporation, the resultant deep red oil was crystallized from a mixture of petrolether and diethylether.

Yield: .3.56 g (83%), mp:70° C.

### EXAMPLE 3

### Preparation of 5-(Dimethylamino)-2-(2,2,2-trifluoroacetyl)penta-2,4-dienenitrile

In a 500 ml three necked round bottom flask equipped with magnetic stirrer, thermometer, dropping funnel and N₂ inlet 8.88 g (121.6 mmol) of N,N-dimethylformamide was dissolved in 350 ml of DCM. The clear solution was cooled to 0° C. under N₂ atmosphere and oxalyl chloride (10.6 ml, 15.43 g, 121.6 mmol) was added dropwise over a period of 30 min. During the addition a white precipitate was formed. The reaction mixture was allowed to warm up to room temperature and stirred for 2 hours. It was cooled again to 0° C. and 1-(vinyloxy)butane (24.32 g, 31.4 ml) was added dropwise keeping the internal temperature below 5° C. The addition took 30 min. Then, the cooling bath was removed and the reaction was stirred for 2 hours under N₂. The pale yellow homogeneous solution was cooled again to −5° C., and 4,4,4-trifluoro-3-oxobutanenitrile (12.82 g, 93.57 mol) was added rapidly followed by the addition of triethylamine (30.7 g, 42 ml, 304 mmol) keeping the temperature below 3° C. The cooling bath was removed and the red reaction mixture was stirred for 5 min. whereupon 300 ml of 1 N HCl was added below 8° C. The resultant two phase mixture was separated; the aqueous phase was extracted with 50 ml of DCM. The combined organic phases were washed twice with 100 ml of water and dried over MgSO₄. After filtration the solvent was removed by vacuum and evaporated again with 200 ml of toluene. The resulted orange crude product was triturated with diethyl ether, filtered, washed with petroleum ether and dried in air.

Yield: 10.39 g yellow crystals (51%), mp:165° C.

### EXAMPLE 4

### Preparation of 5-(Dimethylamino)-2-(2,2,2-trifluoroacetyl)penta-2,4-dienenitrile

3-(dimethylamino)acrylaldehyde (3.54 g, 35.7 mmol) in acetic anhydride (7.78 ml, 82 mmol) was added to 4,4,4-trifluoro-3-oxobutanenitrile (3.00 g, 21.89 mmol) in a 20 ml pear flask to give a brown solution. The reaction mixture was then stirred for 10 min. TLC showed the reaction to be completed. The dark solution was dissolved in DCM. The organic phase was washed with 1 N HCl solution, water and dried over MgSO₄. After filtration and evaporation, the resultant deep brown crystallines were filtered from diethylether and recrystallized from hot isopropanol (ca. 35 ml).

Yield: .2.25 g (47%), mp:165° C.

### EXAMPLE 5

### Preparation of Ethyl 2-(trifluoromethyl)nicotinate

In a 1 L one necked round bottom flask equipped with magnetic stirrer and reflux condenser, ethyl 5-(dimethylamino)-2-(2,2,2-trifluoroacetyl)penta-2,4-dienoate (80.50 g. 303.8 mmol) was dissolved in a mixture of methanol (700 ml) and 25% aq. ammonia solution (240 ml). The reaction mixture was heated to 70° C. for 20 min. Whereupon it was cooled to room temperature and methanol was removed by evaporation under vacuum. The residue was diluted with water (200 ml) and extracted with diethyl ether (2×300 ml). The organic phase was washed with brine (200 ml), dried over MgSO₄ and filtered. Evaporation of the solvent under vacuum gave 65.0 g of red oil. The crude product was sufficiently pure for the next step.

However, to improve the purity of the ester, after drying over Mg SO₄ filtration was performed through a short silica gel plug and the filtrate was then evaporated to leave 59.9 g of a pale yellow mobile oil (90%).

### EXAMPLE 6

### Preparation of 2-(trifluoromethyl)nicotinonitrile

In a 500 ml one necked round bottom flask equipped with magnetic stirrer and reflux condenser, (Dimethylamino)-2-(2,2,2-trifluoroacetyl)penta-2,4-dienenitrile (13.063 g. 59.92 mmol) was suspended in a mixture of methanol (250 ml) and 25% aq. ammonia solution (23 ml). The reaction mixture was heated to 60° C. for 3.5 hours. Whereupon it was cooled to room temperature and methanol was removed by vacuum. The residue was diluted with water (100 ml) and extracted with diethyl ether (100 ml). The organic phase was dried over MgSO₄ and filtered. Evaporation of the solvent under vacuum gave 10.15 g of red oil. The crude product could be used without any further purification. However, to improve purity the red oil was chromatographed in DCM and homogenous fractions were pooled and evaporated to leave 6.7 g of colourless mobile oil (65%).

### EXAMPLE 7

### Preparation of 3-cyano-2-(trifluoromethyl)pyridine 1-oxide

9.95 g (0.058 mol) of 2-(trifluoromethyl)nicotinonitrile was dissolved in 300 ml of DCM in a 500 mL three necked round bottom flask equipped with magnetic stirrer, thermometer, dropping funnel and N₂ inlet. UHP (54.38 g, 0.578 mol, 5.5 equiv.) was added to the above orange solution in one portion. The reaction was cooled to 0° C. and TFAA (115.4 g, 0.550 mol, 77.6 ml, 9.5 equiv.) was added dropwise keeping the internal temperature below 5° C. After addition of ca. 20 ml of TFFA the reaction mixture started becoming decolorized. During this period the reaction was slightly exothermic. The total addition time was ca. 20 min. The cooling bath was removed and the reaction was stirred for 16 h. under N₂ atmosphere. Then the white precipitate (unreacted UHP) was filtered off from the colorless reaction mixture. The reaction mixture was washed with small amount of DCM. The mother liquor was cooled by external ice-bath and was carefully quenched by addition of Na₂S₂O₅ solution keeping the internal temperature below 25° C. The reaction mixture was stirred for additional 30 min. Formation of another precipitate (CF₃—(CO)NH—(CO)—NH₂) was observed and this was filtered off and the filter cake was washed with small amount of DCM. The two phases were separated. The aqueous phase was extracted with 50 ml of DCM. The combined organic phases were washed twice with 100 ml of water and dried over MgSO₄. After filtration, the mixture was evaporated under vacuum giving yellowish oil. The crude product was uncrystallized on standing in the freezer overnight. It was added to a column and eluted with DCM:MeOH=95:5.Yield: 5.5 g, (61%).

### EXAMPLE 8

### Preparation of (Z)-3-(N′-hydroxycarbamimidoyl)-2-(trifluoromethyl)pyridine 1-oxide

3-Cyano-2-(trifluoromethyl)pyridine 1-oxide (5.797 g, 30.835 mmol) was dissolved in 96% EtOH (174 ml) in a 500 ml one necked round bottom flask. The solution was treated with 50% aq. NH₂OH solution (47 ml, 25 equiv.). The reaction was stirred for 5 hours at room temperature. Then the mixture was evaporated to dryness. The crude crystals were boiled with ca. 30 ml of IPA. The mixture was cooled by ice-bath, filtered and washed with a small amount of IPA. The product was dried under vacuum over P₂O₅. Yield: 5.52 g (81%) m.p.:230° C.

1. Compound of the formula (I) 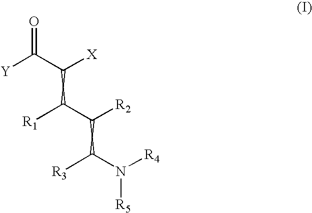
wherein X represents CN, COOR, wherein R represents hydrogen or a carboxyl protecting group, CONR′₂, wherein R′ represents hydrogen or C₁-C₆ alkyl, or nitro; R₁, R₂, R₃, R₄, R₅ independently of each other represent hydrogen, C₁-C₆ alkyl, halogen, trifluoromethyl, cyano, nitro, substituted aryl or substituted heteroaryl group; Y represents hydrogen, C₁-C₆ alkyl, C₃-C₇ cycloalkyl, C₇-C₁₃ alkaryl, trifluoromethyl, cyano, nitro, substituted aryl or substituted heteroaryl group; and wherein the stereochemically unspecified double bonds in the above formula (I) represent either the E,E; E,Z; Z,E or Z,Z configuration.

2. Compound according to claim 1, wherein the substituents R₁ and X as well as R₂ and R₃ are each in trans-positions.

3. Compound according to claim 1, wherein R is hydrogen, C₁-C₆ alkyl, C₃-C₇ cycloalkyl, or C₇-C₁₃ alkaryl.

4. Compound according to claim 1, wherein R is methyl, ethyl, n-propyl, iso-propyl, n-butyl, tert-butyl, phenyl, benzyl, 4-nitro benzyl, 4-bromo benzyl, 4-methoxy benzyl, diphenylmethyl, or trichloroethyl.

5. Compound according to claim 1, wherein Y is trifluoromethyl.

6. Process for the preparation of compounds according to formula (I) as defined in claim 1, comprising a reaction between a compound of general formula (II), 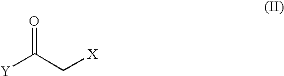
and a compound of formula (III), 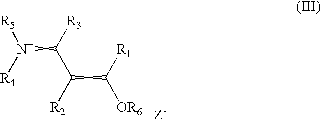
wherein, R₆ is C₁-C₆ alkyl; and Z⁻ represents a suitable counter ion; and wherein the stereochemically unspecified double bonds in the above formula (III) represent the E,E; E,Z; Z,E or Z,Z configuration.

7. Process according to claim 6, wherein the reaction is carried out in the presence of a base.

8. Process according to claim 7, wherein the base is selected from trimethylamine, triethylamine, diisopropylethylamine, pyridine, lutidine, N,N-dimethyl piperidine, N-methyl-pyrrolidine, 1,5-diazabicyclo[4.3.0]non-5-ene, 1,8-diazabicyclo-[5.4.0]undec-7-ene or sodium hydride.

9. Process according to claim 6, wherein the reaction is conducted in the presence of a solvent.

10. Process as claimed in claim 9, wherein the solvent is selected from dichloromethane, 1,2-dichloroethane, 1,1,2,2-tetrachloroethane or chlorobenzenes.

11. Process for the preparation of compounds according to formula (I) as defined in claim 1, comprising a reaction between a compound of formula (II), 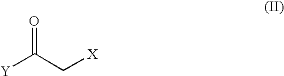
and a compound of formula (IV), 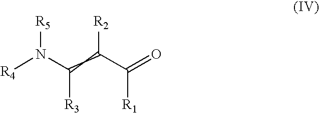
wherein the stereochemically unspecified double bond in the above formula (IV) represents either the E or Z configuration.

12. Process according to claim 12, wherein the reaction is carried out in the presence of an activating agent.

13. Process according to claim 12, wherein the activating agent is acetic anhydride, trifluoroacetic anhydride, methylsulfonic anhydride, phenylsulfonic anhydride, succinic anhydride, phthalic anhydride or an acid chloride.

14. Process according to claim 1, wherein a base is used.

15. Process according to claim 14, wherein the base is selected from trimethylamine, triethylamine, diisopropylethylamine, pyridine, lutidine, N,N-dimethyl piperidine, N-methyl-pyrrolidine, 1,5-diazabicyclo[4.3.0]non-5-ene, 1,8-diazabicyclo[5.4.0]undec-7-ene or sodium hydride.

16. Process for the preparation of compound of the formula (V) 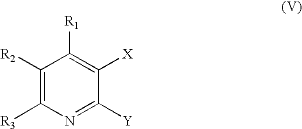
comprising the step of cyclising in the presence of a source of ammonia a compound of formula (I): 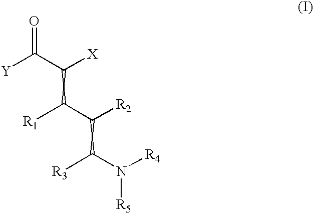
wherein X represents CN, COOR, R represents hydrogen or a carboxyl protecting group, CONR′₂, wherein R′ represents hydrogen or C₁-C₆ alkyl, or nitro; R₁, R₂, R₃, R₄ and R₅ independently of each other represent hydrogen, C₁-C₆ alkyl, halogen, trifluoromethyl, cyano, nitro, substituted aryl or substituted heteroaryl group; Y represents hydrogen, C₁-C₆ alkyl, C₃-C₇ cycloalkyl, C₇-C₁₃ alkaryl, trifluoromethyl, cyano, nitro, substituted aryl or substituted heteroaryl group.

17. Process according to claim 16, wherein the ammonia source is an aqueous ammonia solution, a non-aqueous ammonia solution, ammonium acetate, ammonium sulfamate or ammonia gas.

18. Process according to claim 16, wherein the cyclisation is carried out under non-aqueous conditions.

19. Compound of the formula (VI) 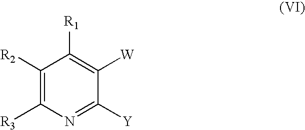
wherein R₁, R₂ and R₃ independently of each other represent hydrogen, C₁-C₆ alkyl, halogen, trifluoromethyl, cyano, nitro, substituted aryl or substituted heteroaryl group; Y represents hydrogen, C₁-C₆ alkyl, C₃-C₇ cycloalkyl, C₇-C₁₃ alkaryl, trifluoromethyl, cyano, nitro, substituted aryl or substituted heteroaryl group; and W represents an amidoxime residue (C(═N—OH)—NH₂), CN, COOR, wherein R represents hydrogen or a carboxyl protecting group, CONR′₂, wherein R′ represents hydrogen or C₁-C₆ alkyl, or nitro.

20. Compound according to claim 19, wherein Y is CF₃ and W is selected from CONH₂ and amidoxime residue (C(═N—OH)—NH₂).

21. Compound of the formula (VII) 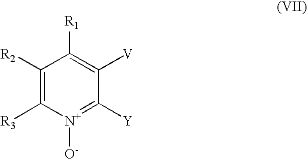
R₁, R₂, and R₃ independently of each other represent hydrogen, C₁-C₆ alkyl, halogen, trifluoromethyl, cyano, nitro, substituted aryl or substituted heteroaryl group; Y represents hydrogen, C₁-C₆ alkyl, C₃-C₇ cycloalkyl, C₇-C₁₃ alkaryl, trifluoromethyl, cyano, nitro, substituted aryl or substituted heteroaryl group; and V represents an amidoxime residue (C(═N—OH)—NH₂), CN, COOR, wherein R represents hydrogen or a carboxyl protecting group, CONR′₂, wherein R′ represents hydrogen or C₁-C₆ alkyl, or nitro.

22. Compound according to claim 21, wherein Y is CF₃ and V is selected from CN, CONH₂, an amidoxime residue (C(═N—OH)—NH₂) and COOR, wherein R is selected from hydrogen, methyl, ethyl, n-propyl, iso-propyl, n-butyl, tert-butyl, phenyl, benzyl, 4-nitro benzyl, 4-bromo benzyl, 4-methoxy benzyl, diphenylmethyl, and trichloroethyl.

23-25. (canceled)
